(19) United States
(12) Patent Application Publication
(10) Pub. No.: US 2025/0332210 A1
(43) Pub. Date: Oct. 30, 2025

(54) Mechanical Cannabinoid Extraction Method and System

(71) Applicant: Clifford Hilpert, Conroe, TX (US)

(72) Inventor: Clifford Hilpert, Conroe, TX (US)

(73) Assignee: Dynamistic Technologies, Conroe, TX (US)

(21) Appl. No.: 18/648,556

(22) Filed: Apr. 29, 2024

(51) Int. Cl. A61K 36/185

(52) U.S. Cl. CPC A61K 36/3482; A61K 2236/37

(57) ABSTRACT

A method and apparatus that allows for the efficient, safe, and odorless extraction of botanical cannabinoids from botanical biomass by utilizing a closed pressing cavity that may optionally include decarboxylation and coextraction features. The closed pressing cavity mitigates parasitic losses of desirable constituents and is a solventless extraction process making it environmentally friendly.

### CROSS-REFERENCE TO RELATED APPLICATIONS

This application claims priority to U.S. Provisional Patent Application No. 63/513,887, filed on Jul. 16, 2023, titled “Improved Mechanical Cannabinoid Extraction Method and System” the entire disclosures of which are incorporated herein by reference.

### BACKGROUND OF THE INVENTION

### Field of the Invention

This invention is directed to a method and system for effecting the extraction of botanical cannabinoids from botanical biomass.

### Description of Related Arts Invention

There are many well-known mechanical and solvent based extraction methods to extract botanical cannabinoids from botanical biomass. For example, mechanically induced forces can be an effective solventless extraction method at certain temperatures and pressures. Supercritical carbon dioxide can be an effective solvent extraction method when utilized at certain temperatures and pressures. Hydrocarbons such as butane and hexane can be an effective solvent extraction method when utilized at certain temperatures and pressures. Ethanol can be an effective solvent extraction method when utilized at certain temperatures and pressures. While all of these can be effective extraction methods for extracting botanical cannabinoids from botanical biomass, utilizing mechanically induced forces at certain temperatures and pressures to effect extraction of botanical cannabinoids from botanical biomass is considered to be the most organic and safe extraction method.

It is well known within the industry that botanical cannabinoids can be extracted from botanical biomass by applying pressure, and in some cases heat, to botanical biomass causing the botanical cannabinoids to migrate from within the compressed botanical biomass. This method of extraction is commonly referred to, and now referred to herein, as “Rosin Pressing”. The realized botanical cannabinoids, in solution or suspension with other extracted botanical constituents, are commonly referred to, and now referred to herein, as “Rosin”. An example of a state-of-the-art rosin press is that manufactured by Pure Pressure of Denver Colorado. To realize rosin utilizing the Pure Pressure rosin press or other state-of-the-art rosin presses, an operator places the botanical biomass into a filter bag. The filter bag is placed in between the pressing plates of the rosin press with the pressing plates being lined with parchment paper and in some cases heated. The rosin press compresses the filter bag between the pressing plates causing the rosin to migrate from within the botanical biomass, through the permeable filter bag, onto the parchment paper. Once the pressing cycle is complete the pressure is released, and the filter bag and parchment paper are removed from the rosin press. The filter bag is then removed from the parchment paper leaving the rosin. The rosin is then removed from the parchment paper, scraping the rosin from the parchment paper by hand, and placed into a suitable storage container. While this method and system does extract the rosin from the botanical biomass, it has several shortcomings including being labor intense, having an open press chamber that can be hazardous when not operated properly, cause parasitic loss of desirable constituents, and create pungent odors.

Therefore, there is a need within the industry for a mechanical force method and system to realize efficient, safe, and odorless extraction of rosin from botanical biomass without parasitic losses of desirable constituents.

### SUMMARY OF THE INVENTION

The present invention overcomes the above noted shortcomings by providing a chamber having at least a first opening for receiving and discharging the botanical biomass, a second opening for discharging the extracted rosin, and a means of reducing the volume of said pressing chamber.

A preferred embodiment of the apparatus could include a pressing chamber that has a cylindrically shaped cavity closed on one end and opened on the other end with the opening being principally the same diameter as the diameter of the cylindrical bore of the cylindrically shaped cavity of the pressing chamber. Additionally, the pressing chamber would include at least one opening located at or near the closed end of the cylindrically shaped cavity of the pressing chamber. A means of reducing the volume of the pressing chamber, for example a movable plug that can be displaced axially along the axis of the cylindrically shaped cavity of the pressing chamber towards the closed end of the cylindrically shaped cavity of the pressing chamber would be included as a feature of the apparatus, as well as, a motive means capable of causing the displacement of the movable plug along the axis of the cylindrically shaped cavity of the pressing chamber with sufficient force to cause the rosin of the botanical biomass to migrate from within the botanical biomass, through the permeable filter bag and subsequently discharged from within the pressing chamber. This preferred embodiment could also include a means of automatically controlling the various parameters utilized to realize efficient, safe, and odorless extraction of rosin from botanical biomass without parasitic losses of desirable constituents.

The following detailed description of the preferred embodiment of the Improved Mechanical Cannabinoid Extraction Method and System is intended as an exemplification of the principals of the invention and not intended to limit the invention to any specific embodiment.

### BRIEF DESCRIPTION OF DRAWINGS

FIG. 1 is a perspective view of the preferred embodiment of the invention depicting the pressing chamber in the pressing position.

FIG. 2 is a section view of the preferred embodiment of the invention.

FIG. 3. Is a perspective view of the preferred embodiment of the invention depicting the pressing chamber in the loading position.

### DETAILED DESCRIPTION OF THE INVENTION

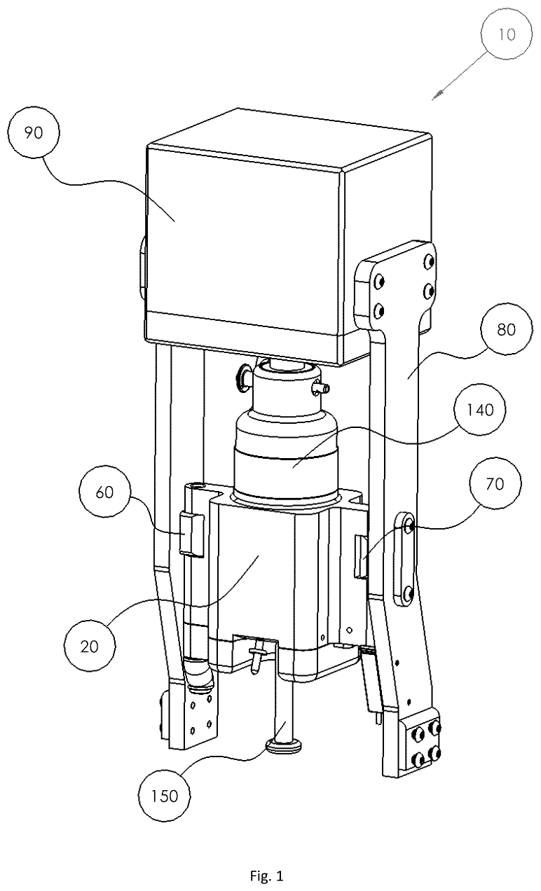

An example of the preferred embodiment of the apparatus of this invention is illustrated in FIG. 1 including pressing chamber 20, chamber support 60, chamber support 70, tie bar set 80, pressing cylinder 90, pressing head 140, and ejector 150.

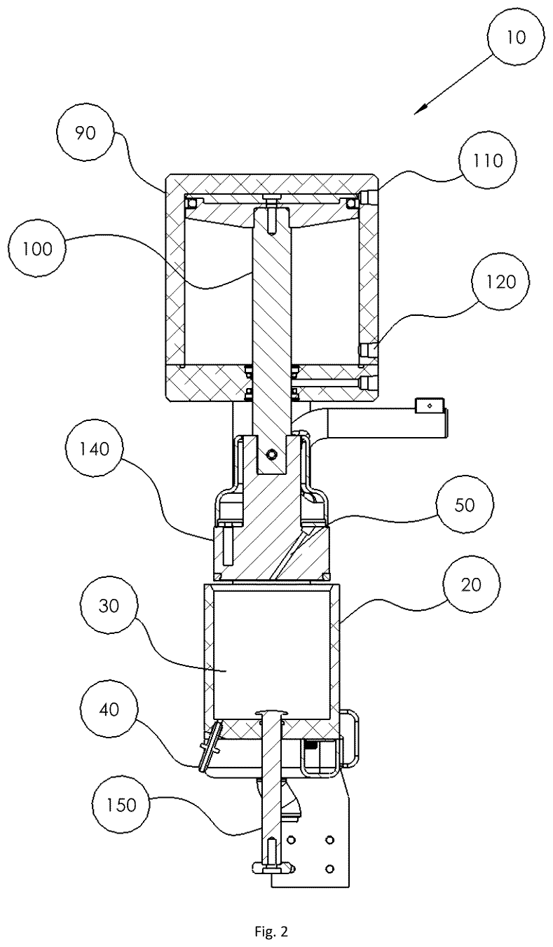

Now describing in detail, with reference to FIG. 2, the preferred embodiment of the apparatus of this invention.

Rosin Press 10 features pressing chamber 20 having a cylindrically shaped cavity, herein referred to as pressing cavity 30. Pressing cavity 30 having a closed end diametrically opposed from an open end, the open end diameter being principally equal to the diameter of pressing cavity 30. Pressing chamber 20 features rosin outlet port 40 located near the closed end of pressing cavity 30 with the opening passing through the wall of pressing chamber 20. Pressing chamber 20 features a heat source to elevate the temperature of pressing chamber 20 above ambient.

Now referring to FIG. 1. Pressing chamber 20 is mechanically coupled to tie bar set 80 via chamber support 60 and chamber support 70. Chamber support 60 features a hinge mechanism allowing pressing chamber 20 to pivot about the axis of the hinge of chamber support 60. Tie bar set 80 is further mechanically coupled to pressing cylinder 90.

Now referring to FIG. 2. Pressing cylinder 90 features a cylindrical bore designed to receive hydraulic piston 100. Pressing cylinder 90 features fluid port 110 and fluid port 120. Fluid port 110 and fluid port 120 are designed to freely pass intensification fluid into and out of the cylindrical bore of pressing cylinder 90. Hydraulic piston 100 is mechanically coupled to pressing head 140. Pressing head 140 can be displaced along the axis of the cylindrical bore of pressing cavity 30. Pressing head 140 features a heat source to elevate the temperature of pressing head 140 above ambient. Pressing chamber 20 features ejector 150.

Rosin press 10 may include a process controller in signal communication with various sensors such as pressure transducers, thermocouples, and safety switches to facilitate various levels of automation.

Another new and unique feature of this invention is the optional ability to coextract where, in addition to all other aspects of the apparatus just described above, an apparatus for providing a suitable coextraction fluid such as olive oil or nitrogen gas can be provided via coextraction inlet port 50 or pressing head 140.

Additionally, a suitable decarboxylation vessel may be connected to rosin outlet port 40.

Now describing in detail two embodiments of the preferred methods of the invention. It can be appreciated that rosin extraction of an ever-different botanical biomass may require different methods to efficiently effect the extraction. In addition, in certain cases such as where the end product is a cream, lotion, or balm, it may be desirable to coextract with an oil or other suitable carrier fluid. In other situations, it may be desirable to coextract with nitrogen gas to eliminate the rosin from being exposed to oxygen during the pressing cycle. In such cases where the end product is to be decarboxylated, Rosin press 10 can optionally be configured to include a decarboxylation vessel mechanically coupled to and in fluid communication with rosin outlet port 40.

The new and unique features of rosin press 10 of this invention are suitable for a wide verity of methods of extractions including, but not limited to, the following examples.

First describing an extraction without a coextraction fluid with reference to FIG. 2.

The pressing cycle consist of subjecting a suitable amount of botanical biomass, contained within a filter bag, to a combination of elevated temperature and pressing pressure, over a period of time, herein referred to as “TPT”, sufficient to effectively displace the rosin from within the botanical biomass into the cavity of pressing chamber 20, then further displaced from within the cavity of pressing chamber 20 via rosin outlet port 40 into a suitable receiving container.

Prior to beginning the pressing cycle, a suitable TPT combination is selected. For example, a certain strain of botanical biomass may yield more rosin from the rosin pressing process where the botanical biomass is exposed to a temperature of approximately 190F, a pressing pressure of approximately 750 psig for a period of time that is approximately 60 sec (TPT=190,750,60). The same botanical biomass may yield a better quality of rosin, but less rosin volume, where TPT=180,650,60. Subsequent to selecting the desired TPT, rosin press 10 is preconfigured to achieve the desired TPT throughout each pressing cycle of the production run.

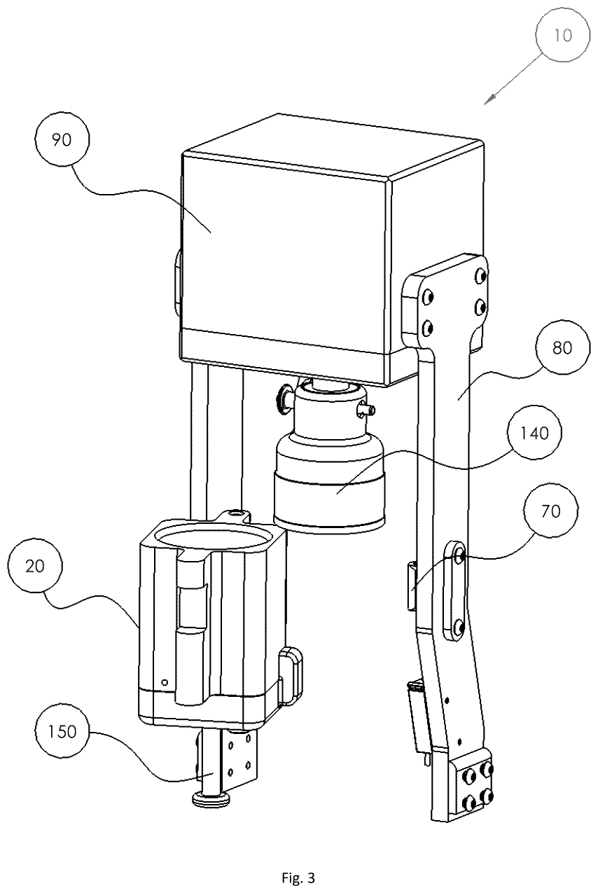

With the TPT of 185,695,65 preconfigured, the pressing cycle begins with pressing head 140 fully retracted from within pressing cavity 30. Pressing chamber 20 is rotated approximately 90 degrees about the axis of the hinge pin of chamber support 60 in a counterclockwise direction to expose the open end of pressing cavity 30 as depicted in FIG. 3, herein referred to as the “Loading Position”. A filter bag containing a suitable amount of botanical biomass is placed into pressing cavity 30. Next pressing chamber 20 is rotated clockwise approximately 90 degrees so as to align the axis of pressing cavity 30 with the axis of pressing head 140, herein referred to as the “Pressing Position”. Subsequently pressing head 140 is displaced towards pressing chamber 20, displacing the botanical biomass in the filter bag, into pressing cavity 30. Pressing head 140 continues to be displaced towards pressing chamber 20 reducing the volume within pressing cavity 30. As the volume of pressing cavity 30 continues to be reduced, the botanical biomass will become more compacted within the confines of pressing cavity 30. As the botanical biomass becomes more compacted it releases the rosin which migrates through the permeable filter bag towards rosin discharge port 40 and then discharged from within pressing cavity 30 via rosin discharge port 40 into a suitable receiving container.

Now describing an extraction with a coextraction fluid. When considering the coextraction process, the pressing cycle consist of subjecting a suitable amount of botanical biomass, contained within a filter bag, and a suitable amount of coextraction fluid, to the desired TPT, sufficient to effectively displace the rosin from within the botanical biomass into pressing cavity 30, where the coextraction fluid and the rosin form a slurry, solution, mixture or otherwise coexist within pressing cavity 30 and then further displaced from within pressing cavity 30 via rosin discharge port 40 into a suitable receiving container.

Prior to beginning the pressing cycle where a coextraction fluid is utilized, a suitable TPT combination is selected, such as the previously selected 185,650,60, to be utilized throughout each pressing cycle of the production run.

With the TPT of 185,650,60 preconfigured the pressing cycle begins with pressing chamber 20 in the loading position. A suitable amount of botanical biomass is placed in a suitable filter bag and placed into pressing cavity 30. Subsequently pressing chamber 20 is rotated into the pressing position and pressing head 140 is displaced towards pressing chamber 20, displacing the botanical biomass, in the filter bag, into pressing cavity 30 and continuing until the point where pressing head 140 has effectively sealed the open end of pressing cavity 30.

At this point in the pressing cycle, a desired amount of coextraction fluid is introduced into pressing cavity 30 via coextraction fluid port 50. Subsequent to the introduction of the coextraction fluid, the pressing cycle continues as previously described. When the coextraction fluid is a liquid, a mixing nozzle may be utilized at the discharge side of rosin port 40 to ensure that the rosin and the coextraction fluid are homogenized.

A suitable decarboxylation vessel may be attached to rosin discharge port 40 to effect decarboxylation of the coextracted rosin. In such case, coextracted rosin being discharged from rosin discharge port 40 is further displaced through a suitable decarboxylation vessel and then into a suitable receiving container. The temperature of the suitable decarboxylation vessel is maintained at approximately 240F. The coextracted rosin is exposed to an elevated temperature for a time period sufficient to effect decarboxylation.

Utilizing the new and unique features of the current invention provides for an efficient, safe, and odorless extraction of rosin from botanical biomass without parasitic losses of desirable constituents with or without a coextraction fluid and with or without decarboxylation.

While a preferred embodiment of the present invention has been described, it is meant as illustrative only and not limiting in scope. A full range of equivalents, many variations and modifications, may be naturally occurring from those skilled in the art after review hereof. For example, the hydraulic actuator providing motive force to pressing head 140 may be replaced with a suitable electrical actuator or a suitable air actuator to provide motive force to pressing head 130. In another variation the motive force may be provided by levers, gears, screws, cams, or other mechanically operated devices. In another example pressing head 130 may be fixed and pressing chamber 20 may be axially displaced to reduce the volume of pressing cavity 30.

What is claimed is:

1. A method for extracting botanical cannabinoids from botanical biomass comprising:
a. introducing botanical biomass into a variable volume cavity; and
b. forcibly reducing the volume of the variable volume cavity.

2. The method of claim 1 further including passing the extract through a decarboxylation vessel prior to collecting the extract.

3. The method of claim 1 further including the introduction of a coextraction fluid during the action of reducing the volume of the variable volume cavity.

4. An apparatus for extracting botanical cannabinoids from botanical biomass comprising:
a. a variable volume cavity; and
b. a means of forcibly reducing the volume of the variable volume cavity.

5. The apparatus of claim 4 further including a decarboxylation vessel.

6. The apparatus of claim 4 further including a coextraction fluid inlet passageway in fluid communication with the variable volume cavity.
